(19) United States
(12) Patent Application Publication
(10) Pub. No.: US 2021/0290800 A1
(43) Pub. Date: Sep. 23, 2021

(54) SANITATION DEVICE

(71) Applicant: James Rudy, Lafayette, IN (US)

(72) Inventor: James Rudy, Lafayette, IN (US)

(21) Appl. No.: 17/208,566

(22) Filed: Mar. 22, 2021

Related U.S. Application Data
(60) Provisional application No. 62/993,591, filed on Mar. 23, 2020.

(51) Int. Cl. A61L 2/18; B05B 1/20; B05B 12/12; B05B 15/14; B60S 3/04

(52) U.S. Cl. CPC A61L 2/18; B05B 1/205; A61L 2202/15; B05B 15/14; B60S 3/04; B05B 12/122

(57) ABSTRACT

This disclosure provides a sanitation device comprising spraying unit and a ramp unit operably linked to one another, as well as methods for preparing and using the same.

### RELATED APPLICATIONS

This application claims priority Ser. No. 62/993,591 filed on Mar. 23, 2020, which is hereby incorporated by reference into this application in its entirety.

### FIELD OF THE DISCLOSURE

This disclosure provides a sanitation device comprising spraying unit and a ramp unit operably linked to one another, as well as methods for preparing and using the same.

### BACKGROUND OF THE DISCLOSURE

There is a need for devices and methods for disinfecting surfaces. This disclosure provides a sanitation device comprising spraying unit and a ramp unit operably linked to one another.

### BRIEF DESCRIPTION OF THE DRAWINGS

FIG. 1. Exemplary ramp and spraying units.

FIG. 2. Exemplary ramp unit and other components.

FIG. 3. Exemplary ramp and spraying units.

FIG. 4. Exemplary spraying unit.

FIG. 5. Exemplary ramp and associated components.

FIG. 6. Exemplary control unit.

FIG. 7. Exemplary subunit of spraying unit.

FIG. 8. Exemplary subunit of spraying unit.

FIG. 9. Exemplary subunit of spraying unit.

FIG. 10. Exemplary subunit of spraying unit.

### SUMMARY OF THE DISCLOSURE

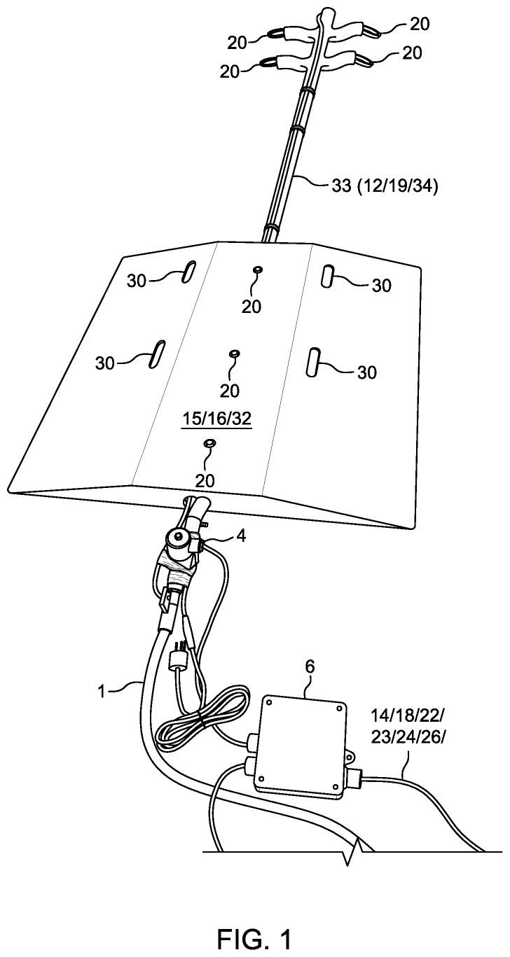

In some embodiments, this disclosure provides a sanitation device comprising spraying unit and a ramp unit operably linked to one another, said device comprising one or more of: conduit (1) (e.g., heated hose), chemical inductor (2), holding tank (for disinfecting solution) (3), electric solenoid (4), 45 degree pipe elbow(s) (7), 15-35 degree angle pipe elbow(s) (8), water supply pipe (9), bushing material (to reduce pipe to proper size) (10), five-way water pipe fitting (11), water flow pipe (12), photo eye sensor (13), PVC (14; lid cover for 24); steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipe; spray tip nozzle(s) (20); T intersection water pipe (21); extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); holes in steel plate (30) (through which water/disinfectant can be sprayed onto underside of vehicle); fork pocket tubes (31); ramp/speed bump component of sanitation station (32) which houses pipe system (33); and/or (34) insulation; and/or as illustrated in any one or more of FIGS. 1-10. In some embodiments, this disclosure provides methods for making such a sanitation device by assembling the components such that water and/or a disinfecting solution can be introduced into the spraying unit and applied to a vehicle traversing the ramp unit of the sanitation device. In some embodiments, this disclosure also provides methods for using the sanitation device of claim 1 to disinfect a vehicle, optionally at least the underside of a vehicle by applying a disinfecting solution to the vehicle as the same traverses the ramp/speed bump component. Other embodiments are also contemplated herein as would be understood by those of ordinary skill in the art.

### DETAILED DESCRIPTION

This disclosure relates to a device and a method associated with the sanitation device that can be used to clean the underside of vehicles entering and exiting a facility. In particular, the sanitation device can be used to clean vehicles entering and exiting a facility for farming and/or processing livestock facilities, and feed mills. The sanitation station can attempt to stop the movement of the following listed diseases and pathogens. In some embodiments, the sanitation device disclosed herein provides for the disinfecting of the vehicles, preferably automatically by detecting the approach of a vehicle and automatically activating the sanitation device to apply a disinfecting solution and/or water to the vehicle (e.g., the underside of the vehicle). This device could be used in any personal or commercial setting, such as but not limited to swine, beef, dairy, poultry, genetics, animal processing facilities, and/or feed mill areas, as well as the human health services industry to assist in controlling disease outbreaks (e.g., hospital settings). In some embodiments, the sanitation device can be used to coat a vehicle in water and sanitizing solution that would destroy organisms, DNA, RNA, and/or other materials associated with various diseases and/or pathogens.

In one embodiment, the sanitation device is a drive-over sanitation station, which disinfects and cleans the underside or undercarriage of any vehicle or implement that drives or passes over it, can be accomplished in three modes of action. In some embodiments, a vehicle approaching the sanitation station can break a beam sending a signal which can in turn activate a solenoid that can allow water to start to flow, in some embodiments by engaging a pump to increase the pressure in the system, causing the water to pull disinfecting solution from a storage tank into the water, which releases from spray nozzles position below and preferably within the ramp, thereby coating the underside of any vehicle that passes over it. The solution can thereby be used to physically remove debris from and disinfectant the underside of the vehicle.

In some embodiments, the core components of the sanitation device include support materials, pipe and plumbing system(s), electronics, and the like. Examples of the same are described below:
1) Pipe and plumbing system (e.g., which forms the spray or spraying unit). Prior to the distribution pipe is a heated hose from the water and/or cleaning (e.g., disinfecting) solution source. After the heated hose is the chemical inductor which is essential for bringing the cleaning solution into the water line, which can be stored in the chemical holding tank. Next in line is an electronic solenoid valve that controls and regulates the flow of water. The water/cleaning solution then flows into the main piping, in some embodiments pressurized by a pump. The pipe and plumbing system can be comprised of various lengths of pipe, pipe fittings, T's, Five-way fittings, 90 degree elbows, 45 degree elbows, 22 degree angles, 10-5 degree angles, other appropriately angled elbows and the like, bushing fittings, and spray nozzles. Electric heated cord can cover every inch of water pipe, insulation surrounding the heat tape and pipe, along with in some embodiments to affix the parts to one another, such that cold temperatures (e.g., below freezing) will not to freeze any parts of the system (e.g., pipes). The pipe and plumbing system is the main delivery system for the sanitizing solution and water.
2) Electronics (used to activate and accomplish spraying): The electronics of the system are an electronic photo eye, electric solenoid valve, optionally an electric motor powering a pump, timer relay module, heated hose, and heated tape. The photo eye can send a signal to a reflective disc, when the beam has been broken between these two components the system can begin the whole process. As such, when the beam is broken, power is sent to the electronic timer relay and the spraying system is engaged.

These components can be positioned within a support and protection system that can effectively be described as a speed bump or ramp, such that in some embodiments, a vehicle can pass over to become cleaned (e.g., sterilized). In a smaller version, the system could be used to clean shoes, boots and the like. In some embodiments, the materials of the speed bump/ramp can include a 2-¼″×12″×120″ flat, 3-¼″×12″×12″×17″ flat sheet cut to right square triangle, 2-¼″×12″×12″ flat, 1-¼″×6″×12″ flat, 2-¼″×2″×10′ flat, 20-¼″×2″×10″ flat, 3-¼″×2″×17″ flat, 2- 3/16″×1½″×1½″×6″ square tubing, 2- 3/16″×2″×6″×12″ square tubing, 2- 3/16×1 ×2×48″ square tubing, 1-6″ channel×120″, 32-⅜″HRR×12″, 4- 3/16″×1½″×1½″×4″, 4- 3/16″×2″×4″. In preferred embodiments, these components can be fixably attached to one another (e.g., welded) to one another creating a gentle slope up and over the piping system. In some embodiments, holes can be cut into the ¼ inch plate to allow sanitizing solution and water to spray and coat undersides of vehicles. In some embodiments, traction bars can be welded in place to provide vehicles traversing the sanitation device sufficient traction. In some embodiments, fork pocket tubes can be welded into the speed bump/ramp, this can make for easy cleaning, maintenance, and movements of the sanitation device.

In some embodiments, the sanitation device comprises one or more channels (e.g., pipe(s)) that carry the water and/or disinfecting solution, typically under pressure, within (e.g., beneath) the “speed bump”. In some embodiments, the piping can range from 3″ diameter to ¼″, and any diameter between those diameters. In some embodiments, the piping system can include different pipe fittings that flow the water, pressure, and disinfecting solution. In some embodiments, the piping system can include T intersections, four- or five-way crosses, and/or reductions bushings. In order to distribute the disinfectant/water out through openings in the speed bump/ramp, the ends of the pipe sections can include a variety of different spraying nozzles and sprinklers. The sprayers are configured to thoroughly coat at least the underside of a car/vehicle/tractor/semi/trailer with pressured, heated water, and sanitation solution (e.g., disinfectant). In some embodiments, the electrical components can include an electric photo eye, electric delay timer, electric solenoid, electric heating tape surrounding the pipe, electric heated hose, optionally a pump, and/or a chemical inducer (e.g., mechanical). In preferred embodiments, the water/disinfectant flows from (in series) the source through a conduit (e.g., hose), an inducer that picks up the disinfecting solution, solenoid, optionally a motor or pump, then the piping and sprayer(s) to coat the underside of car/vehicle/tractor/semi/trailer.

In some embodiments, the sanitation device includes an electronic solenoid valve that can hold and release the flow of water. Once the solenoid is released, sanitizing solution will flow through the piping system (in some embodiments, under pressure applied by a pump). In some embodiments, as vehicle approaches the sanitation station, a photo eye sensor (13) can send and electronic signal back to a multifunction timer relay (23). This timer relay then sends power simultaneously to an electronic motor that powers a pump (6) using an electronic solenoid (4). These can run for a programmed amount time, e.g., sufficient to clean the entire underside of a vehicle traversing the ramp. The cycle is then completed and the system reset.

In some embodiments, heat tape can be wrapped around the pipe to ensure the pipes and/or fittings do not freeze, in some embodiments including insulation (and, in some embodiments, tape can be used to affix the parts to one another), and/or the photo optic eyes can be used to send signal from the power delayed timer to the solenoid. Such components can be configured as follows: the steel can form a ramp/speed bump device with the piping run through the interior and middle of it; the pipe can be wrapped in heat tape and/or an insulated aluminum foil tape; the pipe can be connected inline to the solenoid, the chemical induction system, and in some embodiments a heated hose. In preferred embodiments, the sanitation device includes two modes of action to accomplish the efficient disinfecting of any vehicle or implement that passes over it. To use the sanitation device, in some embodiments, the vehicle can be driven over the ramp to activate the spraying and disinfecting components of the sanitation device. Ultimately, at the conclusion of these steps, anything that passes over the sanitation device (e.g., traverses over the ramp) can be cleaned and disinfected from any potentially dangerous infectious and/or other undesirable material by the chemical cleaning and disinfecting agent, along with in some embodiments the use of water as a rinse.

In some embodiments, a hose (e.g., heated hose) can be connected directly in-line to the solenoid, chemical inducer, optionally a pump and electric motor, and finally the pipe that runs through the ramp/speed bump, below the drive-over portion (i.e., the ramp per se). the photo eye sensor can be connected to the furthest point of the speed bump and can connect to the power delay timer, that can then move to the solenoid to release water pressure (e.g., applied by a pump) thereby moving the solution through the piping system. In some embodiments, a car/vehicle/tractor/semi/trailer/object moves toward the sanitation station, drives over the speed bump/ramp, at which time the photo eye triggers the delayed timer to run for an appropriate amount of time (e.g., 10-20 seconds), releasing the solenoid and allowing water to flow freely from the garden hose past the solenoid through the chemical induction system. A mechanical system could be used in place of the photo eye trigger. Once the solenoid is released the water pressure can draw the chemical cleaning solution out to the storage container into the line and eventually out of the nozzles onto the underside of the car/vehicle/tractor/semi/trailer, preferably completely coating the underside of the object (e.g., vehicle) in a disinfecting solution to, e.g., eliminate harmful pathogens, viruses, DNA, RNA, potentially dangerous bio-hazardous material that may be present on the object. In some embodiments, a heated hose can connect to the sanitation station to maintain a temperature of above freezing, in conjunction with heat tape wrapped around the pipe, bushings, spray nozzles, misters, solenoids, and chemical induction system, to ensure operation in cold temperatures.

In some embodiments, in addition to the undercarriage cleaning system, a 360-degree coverage system that would essentially coat and cover not only the underside of a vehicle/semi/trailer/car/tractor/object but also the top, sides, and all potential surfaces. Ultraviolet light may also be added to the system to more effectively sanitize and disinfect all potential areas of any object or vehicle/semi/trailer/car/tractor/implement. In some embodiments, a pump and/or motor can be included to boost the pressure moving the solution through the whole system.

In some embodiments, the sanitation device disclosed herein can be portable such that it can moved from one location to another. For instance, a farm may have multiple entrances and/or exits to which the sanitation device can be moved when the same are open for use, or the sanitation device can be moved to another location (e.g., farm).

In some embodiments, the sanitation device can include some or all of the following, as illustrated in FIGS. 1-10: conduit (1) (e.g., heated hose), chemical inductor (2), holding tank (for disinfecting solution) (3), electric solenoid (4), optionally pump (5), optionally motor (6) (e.g., electric), 45 degree pipe elbow(s) (7), 15-35 degree angle pipe elbow(s) (8), water supply pipe (9), bushing material (to reduce pipe to proper size) (10), five-way water pipe fitting (11), water flow pipe (12), photo eye sensor or preferably pressure sensor/signal relay (13) (e.g., the sensing portion thereof (e.g., closed hose) is positioned in front of and/or behind ramp (32) to signal an approaching vehicle and engage and/or disengage the system), PVC (14; lid cover for 24); steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipe; spray tip nozzle(s) (20); T intersection water pipe (21); extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); holes in steel plate (30) (through which water/disinfectant can be sprayed onto underside of vehicle); fork pocket tubes (31); ramp/speed bump component of sanitation station (32) which houses pipe system (33). Insulation (34) can also be included to prevent the conduit (e.g., pipe(s)) from freezing in colder weather. Variations of such embodiments are also contemplated as would be understood by those of ordinary skill in the art.

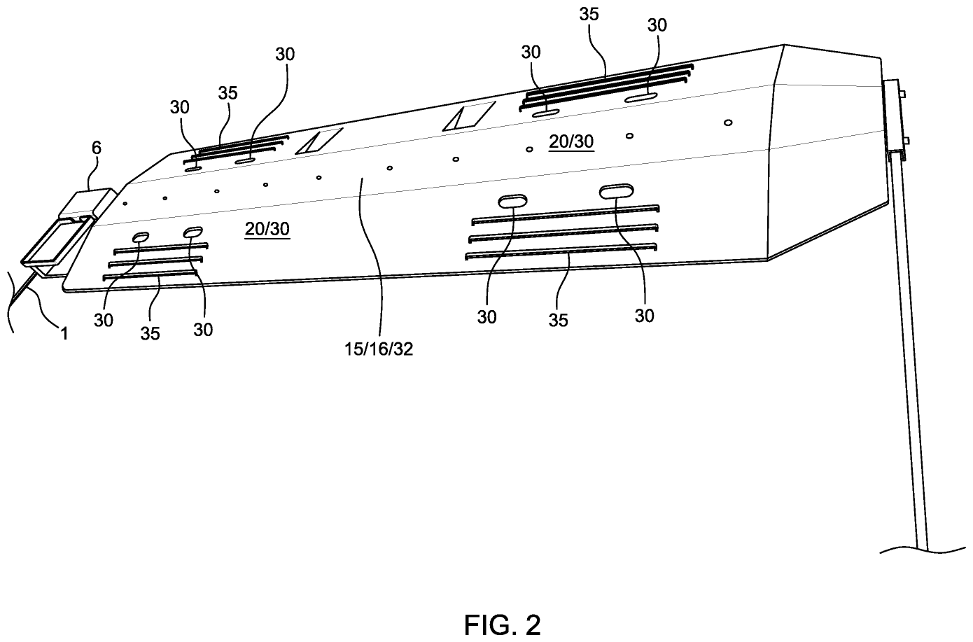

An illustrative embodiment of the sanitation device of this disclosure is provided by FIG. 1, which shows ramp/speed bump component of sanitation station (32) which housing pipe system (33) (one half of ramp/speed bump component (32) is shown with pipe system 33 extending therefrom), including holes in steel plate (30) (steel plate is also referred to herein as a ramp over which a vehicle traverses in operation of the sanitation device) through which fluid (preferably water/disinfectant) can be sprayed onto underside of vehicle, and spray tip nozzles 20. FIG. 2 illustrates ramp/speed bump component 32, within which pipe system 33 is positioned, conduit 1, holes in steel plate 30, and treads (34).

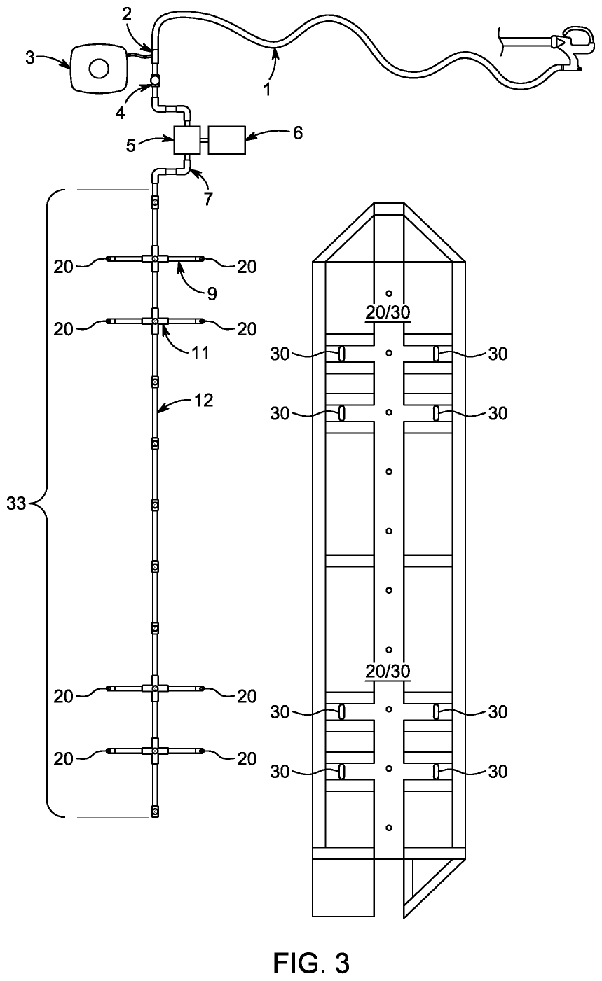

FIG. 3 illustrates an embodiment of the sanitation device including a spraying unit including conduit (1) (e.g., heated hose) supplying water/disinfectant, chemical inductor (2), holding tank (for disinfecting solution) (3), electric solenoid (4), optionally pump (5), optionally motor (6) (e.g., electric), angled (e.g., 45 degree) pipe elbow(s) (7), water supply pipe (9) and water flow pipe (12), spray tip nozzle(s) (20); and the ramp which overlays the spraying unit (e.g., the spraying unit is positioned underneath the ramp) including holes therein (30) through which water/disinfectant can be sprayed onto underside of vehicle.

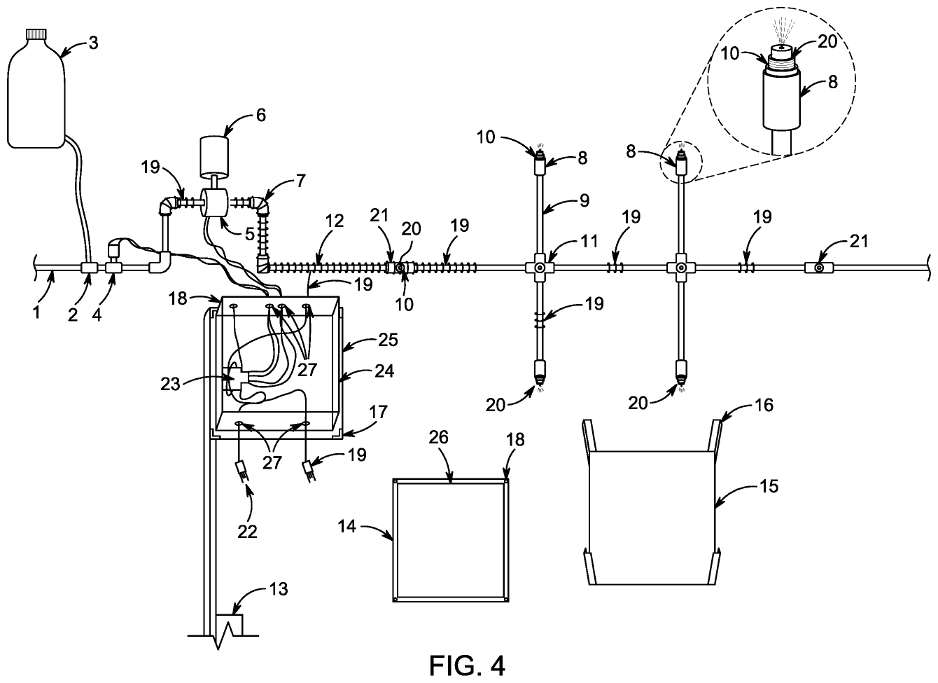

FIG. 4 illustrates an embodiment of the spraying unit including conduit (1) (e.g., heated hose), chemical inductor (2), holding tank (for disinfecting solution) (3), electric solenoid (4), optionally pump (5), optionally motor (6) (e.g., electric), 45 degree pipe elbow(s) (7), 15-35 degree angle pipe elbow(s) (8), water supply pipe (9), bushing material (to reduce pipe to proper size) (10), five-way water pipe fitting (11), water flow pipe (12), photo eye sensor (13), PVC (14; lid cover for 24); steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipes; spray tip nozzle(s) (20); T intersection water pipe (21); extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); which can be housed (e.g., positioned) within ramp/speed bump component of sanitation station (32) (not shown in FIG. 4).

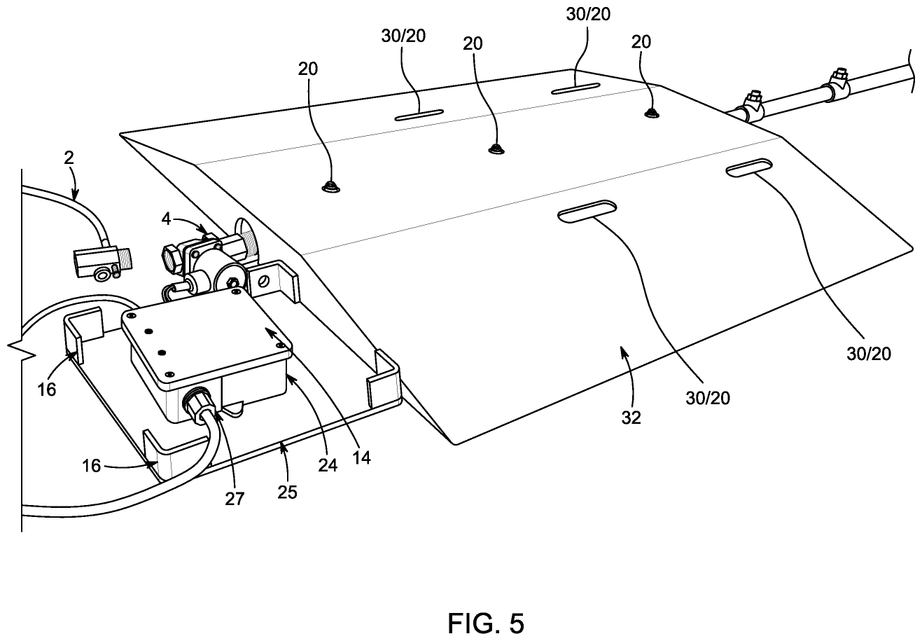

FIG. 5 illustrates an embodiment of the ramp and certain other components, including chemical inductor (2), electric solenoid (4), PVC (14; lid cover for 24); steel angle iron (16; legs for steel lid to protect PVC box); small steel angle iron (17; holds 15 and 16 in place); spray tip nozzle(s) (20) (positioned to eject fluid therefrom though holes in steel plate, also referred to herein as a ramp (30), the holes being positioned to allow fluid to exit nozzles (20) such that the fluid can be ejected therefrom); PVC conduit box (24); plate and base (25; supporting 24); and, grommet(s) (27) (to keep cables watertight from outside vs inside of 24). This embodiment also preferably includes at least one photo eye sensor or preferably pressure sensor/signal relay (13) for engaging and/or disengaging the system.

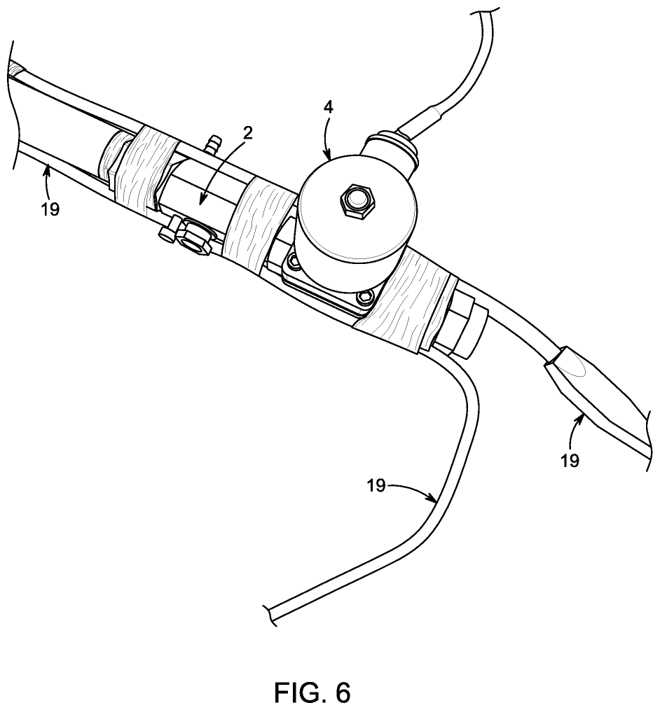

FIG. 6 illustrates an embodiment comprises certain components of the sanitation device, including chemical inductor (2), electric solenoid (4), and heated cord (19) to wrap and cover pipes.

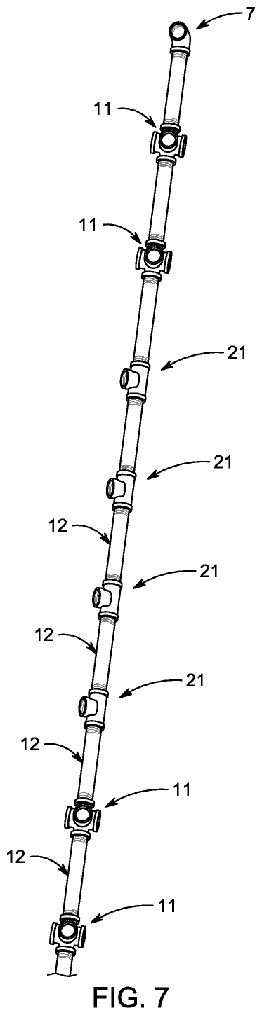

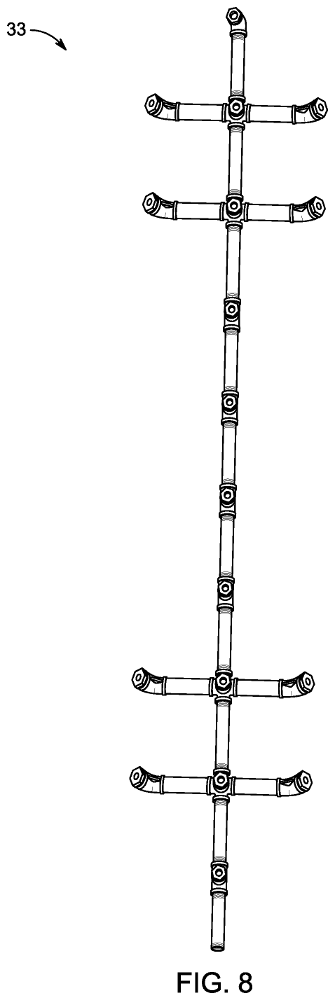

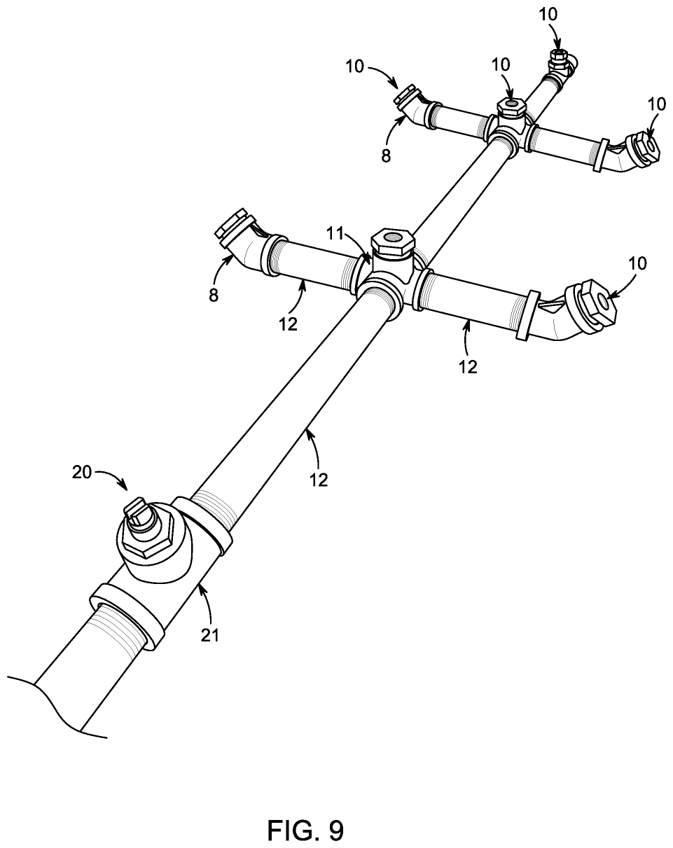

FIG. 7 illustrates an embodiment of the spraying unit showing 45-degree pipe elbow(s) (7), five-way water pipe fitting (11), water flow pipe (12), and T intersection water pipe (21). Pipe system (33) is also illustrated in FIG. 8. FIG. 9 illustrates an embodiment of the spraying unit showing 15-35 degree angle pipe elbow(s) (8), bushing material (to reduce pipe to proper size) (10), five-way water pipe fitting (11), water flow pipe (12), and spray tip nozzle(s) (20). FIG. 9 illustrates an embodiment of the spraying unit showing five-way water pipe fitting (11), water flow pipe (12), heated cord (19) to wrap and cover pipes and spray tip nozzles (20).

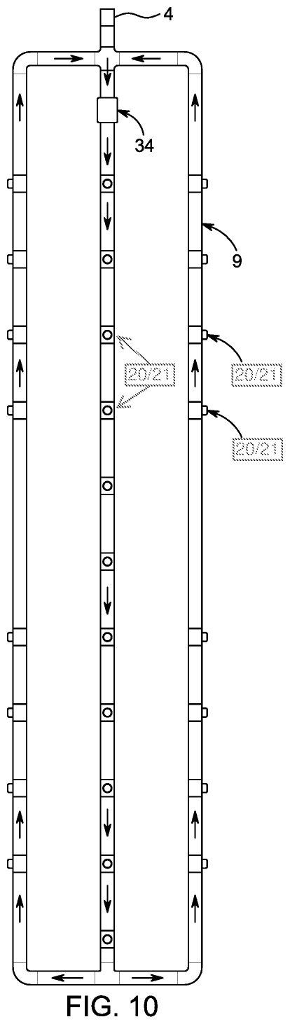

A preferred embodiment is illustrated in FIG. 10. FIG. 10 represents a preferred piping organization layout that could be positioned under steel plate / ramp (e.g., part 32 in FIG. 5), and includes electric solenoid (4), fluid supply pipe (9), heat pump and/or dual heat-pump (34) (e.g., heating the fluid to 170-180° F. (e.g., between freezing and boiling, such as between about 32 and about 112° F.), multiple spray tip nozzle(s) (20/21) (positioned to eject fluid therefrom though holes in steel plate/ramp (30), the holes being positioned therein to allow fluid to exit nozzles (20/21) such that the fluid can be ejected therefrom). The arrows show the flow of fluid through the system as would be understood by those of ordinary skill in the art.

The embodiments illustrated in FIGS. 1-10 can be combined as desired by the user. For instance, the spraying unit may be used with the ramp section in any of the configurations illustrated in FIGS. 4 and 7-10.

Other embodiments are also contemplated herein as would be understood by those of ordinary skill in the art.

The disinfecting solution that can be sprayed from the sanitation device can be any that disinfects, e.g., effectively sterilizes, the vehicle that is treated. Such disinfecting solutions can include, e.g., a bleach-based solution, Tek-Trol Disinfectant cleaner Concentrate by Bio-Tek Industries, Ag- Forte, Synergize, C.O.P. foot pan powder, Activator plus Agricultural Waste Inoculant, Neogen Rodenticide D-Dc&R disinfectant, Zep All-in-1 Pressure washer concentrate, Hanna cleaning & Disinfection solution for dairy products, Virkon S, Virucide by Lanxess, Spray Nine multipurpose Cleaner, EZ-Clean, Chlorhexidine Diacetate Disinfectant by Zoetis, Simple green, EG Ag solutions Synbiont Agricultural wash for large animals, Nixall Farm home ranch cleanser & Deodorizer, Casco QAD10-10% solution general disinfectant, Nolvasan S Disinfectant, DC & R Disinfectant, and/or the like, and/or combinations thereof. It is preferred that such disinfecting solution can eliminate (e.g., kill or at least disable and/or remove from the vehicle): organisms causing bovine, poulty and/or swine diseases including but not limited to anaplasmosis, acetonaemia, anthrax, bluetongue, botulism, BVD, BSE, bovine anaemia, bovine babesiosis, blackleg, bloat, bovine trichomoniasis, brucellosis, calf diphtheria, calf pneumonia, calf scour, cold cow syndrome, cryptosporidiosis, coccidiosis, digital dermatitis, displaced abomasum, epizootic hemorrhagic disease, foot rot, foot and mouth, joint iii, johnes, hypermagnesaemia, ibr, leptospirosis, lice, listeriosis, mange, new forest eye, neosporosis, necrotic enteritis, photosensitisation, pruritus/pyrexia/haemorrhagic syndrome, pseudocowpox, peri-weaning diarhheoa, rumen acidosis, ritaviral diarrhoea, rift valley fever, rumen acidosis, rotaviral diarrhea, ringworm, rabies, schmalenberg, samonella, thrombosis, tetanus, tb, trypanosomosis, traumatic reticuliti, vibriosis, wooden tongue); poultry diseases; bacterial diseases such as Escherichia coli infections, salmonelloses, paratyphoid infections, fowl cholera, riemerella anatipestifer infections, mycoplasma, necrotic enteritis, cholangiohepatitis in broiler chickens, gangrenous dermatitis, botulism, avian tuberculosis; viral diseases such as viral inclusion body hepatitis, haemorrhagic enteritis of turkeys, egg drop syndrome-1976, adeno virus pneumonia, Circo virus, adenovirus group I-associated infections, infectious bursal disease (gumboro), infectious bronchitis (ib), laryngotracheitis, swollen head syndrome, infectious encephalomyelitis, Newcastle disease, fowl pox, reovirus infections, coronavirus, influenza, virus-induced neoplastic diseases Marek's disease, lymphoid leukosis, myelocytomatosis, erythroblastosis, adenocarcinomatosis, Circo virus 2 (post weaning multisystemic wasting syndrome pmws, herpes virus, Aujezky's disease (ad)—pseudorabies (pr), cytomegalovirus, body rhinitis, iridovirus, African swine fever, papovirus, genital papilloma, parvovirus, porcine parvovirus, pox virus, swine pox, arbovirus, Japanese b. encephalitis, aterivirus, porcine reproductive and repiratory syndrome (prrs), calicivirus, vesicular exanthema, cardiovirus, encephalomyocarditis, coronavirus, vomiting wasting disease (hey), porcine respiratory corana virus (prcv), transmissible gastro-enteritis (tge), porcine epidemic diarrhoea (ped), orthomyxovirus, influenza, paramyxovirus, pneumonia, blue eye disease, pestivirus, classical swine fever, picornavirus, foot and mouth disease, techen/talfan disease, smedi, swine vesicular disease, reovirus, rotavirus enteritis, rhabdo virus, rabies vesicular stomatitis); parasitic disease such as coccidiosis, histomonosis, ascaridiosis, raillietinosis, knemidokoptosis, mycoses and mycotoxicoses, aspergillosis, aspergillus granulomatous dermatitis, aflatoxicosis, candidiasis, fusariotoxicoses; actinobacillus pleuropneumonia, anthrax, brucellosis, cystits (e. suis), E. coli scour, enzootic pneumonia, erysielas, greasy pig disease (Staphylococcus), mastitis (Klebsiella), rhinitis, pasteurella, salmonella, streptococcal meningitis, swine dysentery, tuberculosis, arthritis (Mycoplasma hyosynoviae), aniacute flaccid myelitis (AFM), anaplasmosis, anthrax, babesiosis, botulism, brucellosis, campylobacteriosis, carb apenem-resistant infection (cre/crpa), chancroid, chikungunya virus infection (chikungunya), Chlamydia, Ciguatera (harmful algae blooms (habs)), Clostridium difficile infection, Clostridium perfringens (epsilon toxin), coccidioidomycosis fungal infection (valley fever), Creutzfeldt-Jacob disease (CJD), transmissible spongiform encephalopathy, cryptosporidiosis (crypto), cyclosporiasis, Dengue, 1,2,3,4 (Dengue fever), diphtheria, E. coli infection, Shiga toxin-producing (stec), Eastern Equine Encephalitis (EEE), Ebola hemorrhagic fever (Ebola), ehrlichiosis, encephalitis, arboviral or parainfectious, enterovirus infection, non-polio (non-polio enterovirus), enterovirus infection, d68 (ev-d68), giardiasis (Giardia), glanders, gonococcal infection (N. gonorrhea), granuloma inguinale, Haemophilus influenza disease, Type b (hib or h-flu), Hantavirus pulmonary syndrome (HPS), hemolytic uremic syndrome (hus), hepatitis A (hep A), hepatitis B (hep B), hepatitis C (hep C), hepatitis D (hep D), hepatitis E (hep E), Herpes, Herpes zoster, zoster vzv (shingles), histoplasmosis infection (hi stoplasmosi s), human immunodeficiency virus/aids (HIV/AIDS), human papillomavirus (HPV), influenza (flu), legionellosis (Legionnaire's disease), leprosy (Hansenss disease), leptospirosis, listeriosis (listeria), lyme disease, lymphogranuloma venereum infection (lgv), malaria, measles, melioidosis, meningitis, viral (meningitis, viral), meningococcal disease , bacterial (meningitis, bacterial), Middle East Respiratory Syndrome coronavirus (MERS-COV), mumps, norovirus, pediculosis (lice, head and body lice), pelvic inflammatory disease (pid), pertussis (whooping cough), plague; bubonic, septicemic, pneumonic (plague), pneumococcal disease (pneumonia), poliomyelitis (polio), powassan, psittacosis (parrot fever), pthiriasis (crabs; pubic lice infestation), pustular rash diseases (small pox, monkeypox, cowpox), Q-fever, rabies, ricin poisoning, rickettsiosis (rocky mountain spotted fever), rubella, including congenital (German measles), salmonellosis gastroenteritis (Salmonella), scabies infestation (scabies), scombroid, septic shock (sepsis), coronavirus (e.g., severe acute respiratory syndrome (SARS), MERS, SARS-CoV-2), shigellosis gastroenteritis (Shigella), smallpox, Staphyloccal infection (e.g., methicillin-resistant (MRSA)), staphylococcal food poisoning, enterotoxin-b poisoning (staph food poisoning), vancomycin intermediate (VISA), vancomycin resistant (VRSA), streptococcal disease, group A (invasive) (Strep A (invasive)), Streptococcal disease, group B (strep-B), streptococcal toxic-shock syndrome (STSS) or toxic shock (TSS), syphilis, primary, secondary, early latent, late latent, congenital, Tetanus infection, tetani (lock jaw), trichomoniasis (Trichomonas infection), Trichonosis infection (trichinosis), tuberculosis (TB), tuberculosis (latent) (LTBI), tularemia (rabbit fever), typhoid fever, Group D, typhus, vaginosis, bacterial (yeast infection), Varicella (chickenpox), Vibrio Cholerae (cholera), vibriosis (Vibrio), viral hemorrhagic fever (Ebola, Lassa, Marburg), West Nile Virus, yellow fever, Yersenia, and/or Zika virus infection. The disinfecting solutions can also be effective in eliminating and/or otherwise deleteriously affecting other organisms and/or diseases, as is contemplated herein and would be understood by those of ordinary skill in the art.

Thus, in some embodiments, for example as illustrated in the preferred embodiments of FIGS. 1-10, this disclosure provides a sanitation device comprising spraying unit and a ramp unit operably linked to one another, said device comprising one or more of: conduit (1) (e.g., heated hose); chemical inductor (2); holding tank (for disinfecting solution) (3); electric solenoid (4); optionally one or more pump(s) (5); optionally one or more motor(s) (6) (e.g., electric motor); 45 degree pipe elbow(s) (7); angle pipe elbow(s) (e.g., angled at about 15 to about 35 degrees) (8); water supply pipe (9); bushing material (to reduce pipe to proper size) (10); five-way water pipe fitting (11); water flow pipe (12); photo eye sensor, or preferably a pressure sensor (13) to trigger operation of the device; PVC (14; lid cover for 24); steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipe; spray tip nozzle(s) (20); T intersection water pipe (21); extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); holes in steel plate (30) (through which water/disinfectant can be sprayed onto underside of vehicle); fork pocket tubes (31); ramp/speed bump component of sanitation station (32) which houses pipe system (33); and/or insulation (34); and/or treads (35); and/or as illustrated in any one or more of FIGS. 1-10. In some embodiments, this disclosure provides methods for making such a sanitation device by assembling the components such that water and/or a disinfecting solution can be introduced into the spraying unit and applied to a vehicle traversing the ramp unit of the sanitation device. In some embodiments, this disclosure also provides methods for using the sanitation device of this disclosure (e.g., as in claim 1) to disinfect a vehicle, optionally at least the underside of a vehicle by applying a disinfecting solution to the vehicle as the same traverses the ramp/speed bump component. In some embodiments, the fluid traversing the system can be heated prior to entering the system (i.e., pre-heated), and/ or re-heated in a recirculation circuit. Other embodiments are also contemplated herein as would be understood by those of ordinary skill in the art.

In some embodiments, this disclosure provides a sanitation device comprising spraying unit and a ramp unit operably linked to one another, said device comprising one or more of: conduit (1) (e.g., heated hose), chemical inductor (2), holding tank (for disinfecting solution) (3), electric solenoid (4), optionally pump (5), optionally motor (6), 45 degree pipe elbow(s) (7), 15-35 degree angle pipe elbow(s) (8), water supply pipe (9), bushing material (to reduce pipe to proper size) (10), five-way water pipe fitting (11), water flow pipe (12), photo eye sensor (13), PVC (14; lid cover for 24); steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipe; spray tip nozzle(s) (20); T intersection water pipe (21); extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); holes in steel plate (30) (through which water/disinfectant can be sprayed onto underside of vehicle); fork pocket tubes (31); ramp/speed bump component of sanitation station (32) which houses pipe system (33); and/or as illustrated in any one or more of FIGS. 1-10. In some embodiments, this disclosure provides methods for making a sanitation device of this disclosure (e.g., preferably as shown in any one or more of FIGS. 1-10, and/or claim 1), the method comprising assembling the components such that water and/or a disinfecting solution can be introduced into the spraying unit and applied to a vehicle traversing the ramp unit of the sanitation device. In some embodiments, this disclosure provides methods for using the sanitation device of this disclosure (e.g., preferably as shown in any one or more of FIGS. 1-10, and/or claim 1) to disinfect a vehicle, optionally at least the underside of a vehicle by applying a disinfecting solution to the vehicle as the same traverses the ramp/speed bump component. Other embodiments are also contemplated herein as would be understood by those of ordinary skill in the art.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. Optional or optionally means that the subsequently described event or circumstance can or cannot occur, and that the description includes instances where the event or circumstance occurs and instances where it does not. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it can be understood that the particular value forms another aspect. It can be further understood that the endpoints of each of the ranges are significant both in relation to the other endpoint, and independently of the other endpoint. Ranges (e.g., 90-100%) are meant to include the range per se as well as each independent value within the range as if each value was individually listed.

All references cited within this disclosure are hereby incorporated by reference in their entirety. Certain embodiments are further described in the following examples. These embodiments are provided as examples only and are not intended to limit the scope of the claims in any way.

While certain embodiments have been described in terms of the preferred embodiments, it is understood that variations and modifications can occur to those skilled in the art. Therefore, it is intended that the appended claims cover all such equivalent variations that come within the scope of the following claims.

What is claimed is:

1. A sanitation device comprising spraying unit and a ramp unit operably linked to one another, said device comprising one or more of: conduit (1) (e.g., heated hose), chemical inductor (2), holding tank (for disinfecting solution) (3), electric solenoid (4), optionally pump (5), optionally motor (6), 45 degree pipe elbow(s) (7), 15-35 degree angle pipe elbow(s) (8), water supply pipe (9), bushing material (to reduce pipe to proper size) (10), five-way water pipe fitting (11), water flow pipe (12), photo eye sensor (13), PVC (14; lid cover for 24);
steel plate (15; lid for 25); steel angle iron (16; legs for steel lid to protect PVC box), small steel angle iron (17; holds 15 and 16 in place); holes (18) to bolt down PVC lid (14); heated cord (19) to wrap and cover pipe; spray tip nozzle(s) (20); T intersection water pipe (21);
extension cord to power unit (22); timer relay (23); PVC conduit box (24); plate and base (25; supporting 24); water-tight gasket (26); grommet(s) (27) (to keep cables watertight from outside vs inside of 24); reflective disk (28) (to send signal back to photo eye); treads (29) (e.g., metal bars ⅜″ round rod); holes in steel plate (30) (through which water/disinfectant can be sprayed onto underside of vehicle); fork pocket tubes (31); ramp/speed bump component of sanitation station (32) which houses pipe system (33); and/or as illustrated in any one or more of FIGS. 1-10.

2. A method for making a sanitation device of claim 1, the method comprising assembling the components such that water and/or a disinfecting solution can be introduced into the spraying unit and applied to a vehicle traversing the ramp unit of the sanitation device.

3. A method for using the sanitation device of claim 1 to disinfect a vehicle, optionally at least the underside of a vehicle by applying a disinfecting solution to the vehicle as the same traverses the ramp/speed bump component.
